(19) United States
(12) Patent Application Publication
(10) Pub. No.: US 2026/0154816 A1
(43) Pub. Date: Jun. 4, 2026

(54) Information Processing Device, Learning Model Creation Device, Information Processing Method, Learning Model Creation Method and Program

(71) Applicant: TEIKYO UNIVERSITY, Itabashi-ku, Tokyo (JP)

(72) Inventor: Toshifumi MIHASHI, Tokyo (JP)

(73) Assignee: TEIKYO UNIVERSITY, Itabashi-ku, Tokyo (JP)

(21) Appl. No.: 19/311,197

(22) Filed: Aug. 27, 2025

(30) Foreign Application Priority Data
Aug. 30, 2024 (JP) 2024-149795

(51) Int. Cl. G06T 7/00; A61B 3/107; A61B 3/12; A61B 3/14

(52) U.S. Cl. CPC G06T 7/0012; A61B 3/107; A61B 3/12; A61B 3/14; G06T 2207/30041

(57) ABSTRACT

An object of the present invention is to acquire, from fundus image information, information indicating a sphericity of an eye for a subject.

An information processing apparatus includes a reception unit configured to receive fundus image information of a subject, a processing unit configured to acquire information indicating a sphericity of an eye of the subject based on a trained model, which is obtained by performing machine learning on a relationship between fundus image information of an eye and information indicating a sphericity of the eye, and the fundus image information of the subject received by the reception unit, and an output unit configured to output the information indicating the sphericity of the eye of the subject acquired by the processing unit.

### BACKGROUND OF THE INVENTION

### Field of the Invention

The present invention relates to an information processing apparatus, a learning model creation apparatus, an information processing method, a learning model creation method, and a program.

### Description of Related Art

It has been announced that a value of a biomarker such as age, gender, smoking history, and HbA1C can be predicted from a fundus image, and many studies have been conducted since then.

A technology is known in which a refraction abnormality is estimated from a retinal fundus photograph (refer to, for example, Avinash V. Varadarajan, Ryan Poplin, Katy Blumer, Christof Angermueller, Joe Ledsam, Peena Chopra, Pearse A. Keane, Greg S. Corrado, Lily Peng, and Dale R. Webster, “Deep Learning for Predicting Refractive Error From Retinal Fundus Images”, Investigative Ophthalmology & Visual Science, June 2018, Vol. 59, No. 7, pp. 2861-2868). Further, a technology is known in which an ocular axial length is estimated based on a fundus image (refer to, for example, Yeonwoo Jeong, Boram Lee, Jae-Ho Ham, and Jaeryung Oh, “Ocular Axial Length Prediction Based on Visual Interpretation of Retinal Fundus Images via Deep Neural Network”, IEEE JOURNAL OF SELECTED TOPICS IN QUANTUM ELECTRONICS, VOL. 27, NO. 4, JULY/AUGUST 2021.).

### SUMMARY OF THE INVENTION

### Problems to be Solved by the Invention

A sphericity is usually measured by a dedicated device based on a basic principle of measuring a magnification of an optical system, which is obtained by combining an eye and an optical system of a measurement device. However, this examination is generally not performed in a health checkup, for example.

A thickness of a retina is obtained from an optical tomographic image of the retina captured by an optical coherence tomography (OCT), and a fundus shape is quantified. With a result of the quantification of the fundus shape, the sphericity of the eye can be obtained. However, it takes time and effort to obtain the sphericity of the eye.

With development of multimodal artificial intelligence (AI), it is possible to predict a response variable from various parameters. Further, an explainable AI technology that can indicate parameters of interest in a case where a prediction is made, in predicting the response variable, is also in the practical stage.

An object of the present invention is to provide an information processing apparatus, a learning model creation apparatus, an information processing method, a learning model creation method, and a program capable of acquiring information indicating a sphericity of an eye from fundus image information for a subject. In this method, a fundus image is acquired for an examination of diabetes or glaucoma in a health checkup or the like, and it is possible to know the sphericity in the examination, which is beneficial.

### Means for Solving Problems

(1) An aspect of the present invention is an information processing apparatus including a reception unit configured to receive fundus image information of a subject, a processing unit configured to acquire information indicating a sphericity of an eye of the subject based on a trained model, which is obtained by performing machine learning on a relationship between fundus image information of an eye and information indicating a sphericity of the eye, and the fundus image information of the subject received by the reception unit, and an output unit configured to output the information indicating the sphericity of the eye of the subject acquired by the processing unit.

(2) In the information processing apparatus according to an aspect of the present invention, the processing unit acquires information indicating an ocular axial length of the eye of the subject based on a trained model, which is obtained by further performing machine learning on a relationship between a fundus image of the eye and an ocular axial length of the eye, and the fundus image information of the subject received by the reception unit, and the output unit outputs the information indicating the ocular axial length of the eye of the subject acquired by the processing unit.

(3) The information processing apparatus according to an aspect of the present invention further includes an extraction unit configured to extract, from a fundus image, a fundus image of a predetermined region including a macula and an optic nerve head based on the fundus image information of the subject, in which the processing unit acquires the information indicating the sphericity of the eye of the subject, based on the trained model and fundus image information of the predetermined region extracted by the extraction unit.

(4) In the information processing apparatus according to the aspect of the present invention, the trained model includes a first trained model obtained by performing machine learning on a relationship between fundus image information of a right eye and information indicating a sphericity of the right eye, and a second trained model obtained by performing machine learning on a relationship between fundus image information of a left eye and information indicating a sphericity of the left eye.

(5) An aspect of the present invention is a learning model creation apparatus including a reception unit configured to receive a data set for learning in which fundus image information of an eye is included as learning data and information indicating a sphericity of the eye is included as supervised data, a processing unit configured to create a learning model by performing machine learning on a relationship between the fundus image information of the eye and the information indicating the sphericity of the eye, using the fundus image information of the eye as an explanatory variable and the information indicating the sphericity of the eye as a response variable, based on the data set for learning received by the reception unit, and an output unit configured to output the learning model created by the processing unit.

(6) In the information processing apparatus according to an aspect of the present invention, the reception unit receives a data set for learning in which the fundus image information of the eye is included as the learning data and information indicating an ocular axial length of the eye is further included as the supervised data, and the processing unit creates a learning model by performing machine learning on a relationship between the fundus image information of the eye and the information indicating the ocular axial length of the eye, using the fundus image information of the eye as the explanatory variable and the information indicating the ocular axial length of the eye as the response variable, based on the data set for learning received by the reception unit.

(7) An aspect of the present invention is an information processing method executed by a computer, the method including receiving fundus image information of a subject, acquiring information indicating a sphericity of an eye of the subject based on a trained model, which is obtained by performing machine learning on a relationship between fundus image information of an eye and information indicating a sphericity of the eye, and the received fundus image information of the subject, and outputting the acquired information indicating the sphericity of the eye of the subject.

(8) An aspect of the present invention is a learning model creation method executed by a computer, the method including receiving a data set for learning in which fundus image information of an eye is included as learning data and information indicating a sphericity of the eye is included as supervised data, creating a learning model by performing machine learning on a relationship between the fundus image information of the eye and the information indicating the sphericity of the eye, using the fundus image information of the eye as an explanatory variable and the information indicating the sphericity of the eye as a response variable, based on the received data set for learning, and outputting the created learning model.

(9) An aspect of the present invention is a program causing a computer to receive fundus image information of a subject, acquire information indicating a sphericity of an eye of the subject based on a trained model, which is obtained by performing machine learning on a relationship between fundus image information of an eye and information indicating a sphericity of the eye, and the received fundus image information of the subject, and output the acquired information indicating the sphericity of the eye of the subject.

(10) An aspect of the present invention is a program causing a computer to receive a data set for learning in which fundus image information of an eye is included as learning data and information indicating a sphericity of the eye is included as supervised data, create a learning model by performing machine learning on a relationship between the fundus image information of the eye and the information indicating the sphericity of the eye, using the fundus image information of the eye as an explanatory variable and the information indicating the sphericity of the eye as a response variable, based on the received data set for learning, and output the created learning model.

### Effect of Invention

According to the present invention, it is possible to provide the information processing apparatus, the learning model creation apparatus, the information processing method, the learning model creation method, and the program capable of acquiring the information indicating the sphericity of the eye from the fundus image information for the subject.

### BRIEF DESCRIPTION OF THE DRAWINGS

FIG. 1 is a diagram showing an example of an information processing apparatus according to the present embodiment.

FIG. 2 is a diagram showing an example of a fundus image of an eye.

FIG. 3 is a flowchart showing an example of an operation of the information processing apparatus of the embodiment.

FIG. 4 is a diagram showing an example of a learning model creation apparatus according to the present embodiment.

FIG. 5 is a flowchart showing an example of an operation of the learning model creation apparatus according to the present embodiment.

FIG. 6 is a diagram showing an example of an information processing apparatus according to Modification Example 1 of the embodiment.

FIG. 7 is a flowchart showing an example of an operation of the information processing apparatus according to Modification Example 1 of the embodiment.

FIG. 8 is a diagram showing an example of an information processing apparatus according to Modification Example 2 of the embodiment.

FIG. 9 is a flowchart showing an example of an operation of the information processing apparatus according to Modification Example 2 of the embodiment.

FIG. 10 is a diagram showing an example of a learning model creation apparatus according to Modification Example 2 of the embodiment.

FIG. 11 is a flowchart showing an example of an operation of the learning model creation apparatus according to Modification Example 2 of the embodiment.

### DETAILED DESCRIPTION OF THE INVENTION

Hereinafter, an information processing apparatus, a learning model creation apparatus, an information processing method, a learning model creation method, and a program according to an embodiment will be described with reference to drawings. Embodiments to be described below are merely examples, and the embodiments to which the present invention is applied are not limited to the following embodiments.

In all the drawings for describing the embodiments, the same reference numerals are assigned to parts having the same functions, and repeated descriptions will be omitted.

Further, the expression “based on XX” referred to in the present application means “based on at least XX”, and also includes a case of being based on another element in addition to Further, the expression “based on XX” is not limited to a case where XX is directly used, and also includes a case of XX subjected to calculation or processing. The term “XX” is an optional element (for example, optional information).

### EMBODIMENT

### Information Processing Apparatus

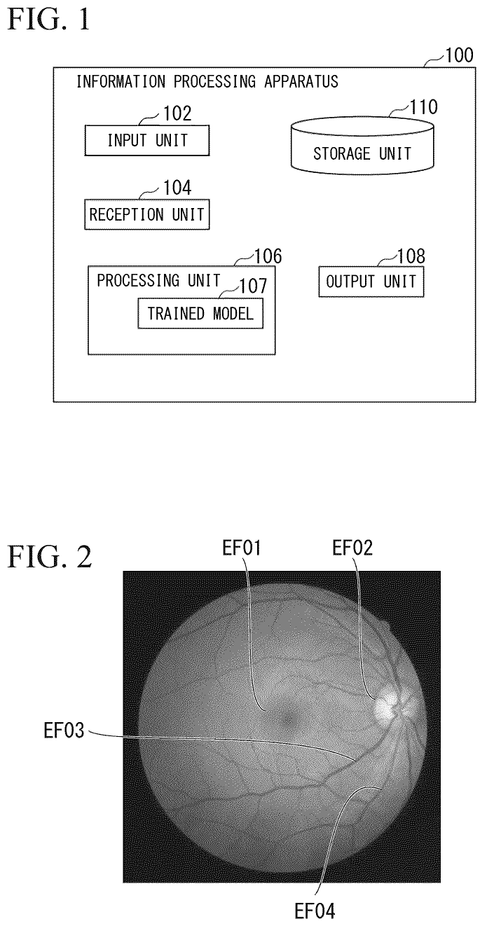

FIG. 1 is a diagram showing an example of an information processing apparatus according to the present embodiment. An information processing apparatus 100 according to the present embodiment receives subject-related information. The subject-related information includes subject identification information and fundus image information of an eye of a subject.

The information processing apparatus 100 acquires information indicating a sphericity of the eye of the subject based on the fundus image information of the eye, which is included in the received subject-related information, and a trained model. The trained model is obtained by performing machine learning on a relationship between the fundus image information of the eye and the information indicating the sphericity of the eye.

The information processing apparatus 100 outputs the subject identification information and the acquired information indicating the sphericity of the eye of the subject.

The information processing apparatus 100 is formed by an apparatus such as a personal computer, a server, a smartphone, a tablet computer, or a computer for industry. The information processing apparatus 100 includes an input unit 102, a reception unit 104, a processing unit 106, an output unit 108, and a storage unit 110.

The input unit 102 receives an input of information. As an example, the input unit 102 may include an operation unit such as a keyboard and a mouse. In this case, the input unit 102 receives the input of information in accordance with an operation performed by a user on the operation unit. As another example, the input unit 102 may receive the input of information from an external apparatus. The external apparatus may be, for example, a portable storage medium. The input unit 102 receives an input of the subject-related information.

The reception unit 104 acquires the subject-related information from the input unit 102. The reception unit 104 acquires the subject identification information included in the acquired subject-related information and the fundus image information of the eye, and receives the acquired subject identification information and fundus image information of the eye.

The fundus image of the eye is obtained by putting light into an eyeball from a pupil and capturing a back (fundus) of the eyeball on a brain side. A macula, an optic nerve head, a retina, and the like can be observed in the fundus image. In general, a color image is often used as the fundus image, but a monochrome image or an image with more than three primary colors may also be used.

Further, an image captured by a simple optical system capable of imaging a macular area and the optic nerve head, which is important for predicting the sphericity or the ocular axial length, may also be useful. For example, even in a case where an optical system of a level of an automatic refractometer is used instead of a full-scale fundus camera used for diagnosis of a disease, the prediction can also be made with an image captured by the optical system.

Further, an image obtained by a scanning optical system can also be used as the image used here.

FIG. 2 is a diagram showing an example of the fundus image of the eye. The fundus image of the eye includes a macula EF01, an optic nerve head EF02, a vein EF03, and an artery EF04. Returning to FIG. 1, the description will be continued.

The processing unit 106 acquires, from the reception unit 104, the subject identification information and the fundus image information of the eye. The processing unit 106 includes a trained model 107. The trained model 107 is obtained by performing machine learning on the relationship between the fundus image information of the eye and the information indicating the sphericity of the eye. A method of creating the trained model 107 will be described below. The processing unit 106 inputs, to the trained model 107, the acquired fundus image information of the eye to acquire the information indicating the sphericity of the eye output by the trained model 107 for the input fundus image information of the eye. The sphericity of the eye is defined as a reciprocal of a focal length represented in meters. In the present embodiment, the sphericity and a refractive index are synonymous.

The output unit 108 acquires, from the processing unit 106, the subject identification information and the information indicating the sphericity of the eye. The output unit 108 outputs the acquired subject identification information and information indicating the sphericity of the eye.

For example, in the output unit 108, the subject identification information and the information indicating the sphericity of the eye may be output by voice, or may be output on a display unit (not shown) in a displayed manner.

Further, the output unit 108 may store the subject identification information and the information indicating the sphericity of the eye in the storage unit 110 in association with each other.

All or a part of the input unit 102, the reception unit 104, the processing unit 106, and the output unit 108 are functional units realized by a processor such as a central processing unit (CPU) executing a program stored in the storage unit 110 (hereinafter, referred to as software functional units).

All or a part of the input unit 102, the reception unit 104, the processing unit 106, and the output unit 108 may be realized by hardware such as a large scale integration (LSI), an application specific integrated circuit (ASIC), or a field-programmable gate array (FPGA), or may be realized by a combination of the software functional units and the hardware.

### (Operation of Information Processing Apparatus 100)

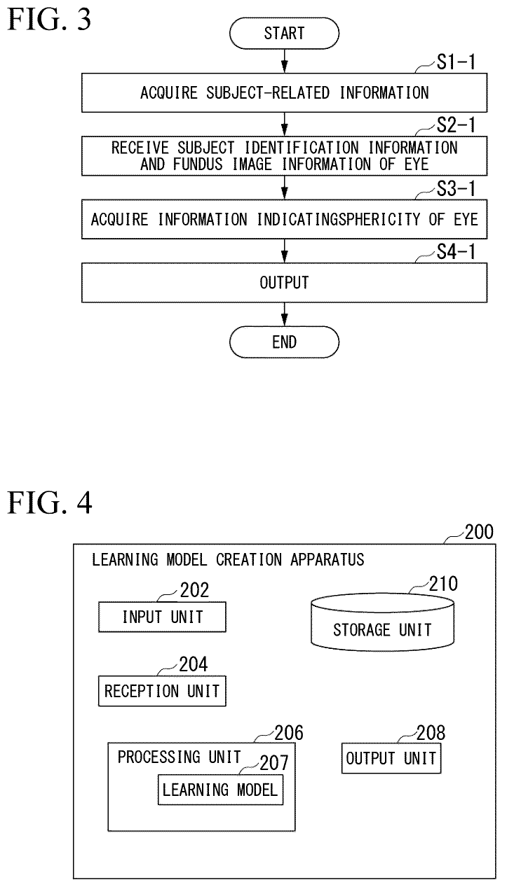

FIG. 3 is a flowchart showing an example of an operation of the information processing apparatus of the embodiment.

### (Step S1-1)

The input unit 102 acquires the subject-related information.

### (Step S2-1)

The reception unit 104 acquires the subject-related information from the input unit 102. The reception unit 104 acquires the subject identification information included in the acquired subject-related information and the fundus image information of the eye, and receives the acquired subject identification information and fundus image information of the eye.

### (Step S3-1)

The processing unit 106 acquires, from the reception unit 104, the subject identification information and the fundus image information of the eye. The processing unit 106 inputs, to the trained model 107, the acquired fundus image information of the eye to acquire the information indicating the sphericity of the eye output by the trained model 107 for the input fundus image information of the eye.

### (Step S4-1)

The output unit 108 acquires, from the processing unit 106, the subject identification information and the information indicating the sphericity of the eye. The output unit 108 outputs the acquired subject identification information and information indicating the sphericity of the eye.

In the above embodiment, the processing unit 106 inputs the fundus image information of the eye of the subject to the trained model 107 in which the fundus image information of the eye of the subject whose sphericity of the eye is actually acquired is already stored to acquire the information indicating the sphericity of the eye of the subject. Hereinafter, the fundus image information of the eye related to the generation of the trained model 107 (subject whose sphericity of the eye is actually acquired and whose fundus image information of the eye is already stored) will be referred to as a model target.

The creation of the trained model 107 will be described. The trained model 107 is created by the learning model creation apparatus. That is, the learning model creation apparatus creates the trained model 107. The information processing apparatus 100 may include the learning model creation apparatus. That is, the information processing apparatus 100 may create the trained model 107.

FIG. 4 is a diagram showing an example of the learning model creation apparatus according to the present embodiment. A learning model creation apparatus 200 according to the present embodiment is formed by an apparatus such as a personal computer, a server, a smartphone, a tablet computer, or a computer for industry.

The learning model creation apparatus 200 trains a learning model (model that is a source of the trained model 107), using a data set for learning in which the fundus image information of the eye of the subject as the model target is used as an input sample and the information indicating the sphericity of the eye of the subject is used as an output sample to create the trained model 107.

For example, the learning model creation apparatus 200 uses an algorithm such as a convolution neural network (CNN), a recurrent neural network (RNN), a long short-term memory (LSTM), a random forest, a support vector machine (SVM), or a neural network to construct the trained model 107. The input sample is data input to an input layer during the training of the learning model. The output sample is data (supervised data) that is a correct answer to be compared with an output value output from an output layer during the training of the learning model.

The learning model creation apparatus 200 includes an input unit 202, a reception unit 204, a processing unit 206, an output unit 208, and a storage unit 210.

The input unit 202 receives an input of information. As an example, the input unit 202 may include an operation unit such as a keyboard and a mouse. In this case, the input unit 202 receives the input of information in accordance with an operation performed by a user on the operation unit. As another example, the input unit 202 may receive the input of information from an external apparatus. The external apparatus may be, for example, a portable storage medium. The data set for learning is input to the input unit 202.

The reception unit 204 acquires the data set for learning from the input unit 202, and receives the acquired data set for learning. The data set for learning includes the input sample and the output sample, and the input sample and the output sample are paired. The data set for learning is configured of a plurality of pairs.

The processing unit 206 calculates, for all pairs, an error between the output value output from the output layer by inputting the input sample to the input layer of the learning model 207 and the output sample (supervised data) corresponding to the input sample, and changes a parameter of the learning model 207 (trains the learning model 207) such that the error is as small as possible to create the trained model 107. For example, the processing unit 206 may perform transfer learning, using the learning model 207 on which pre-training using data such as ImageNet is performed.

The trained model 107 created as described above is received, from the output unit 208, by the information processing apparatus 100 via a network or a medium, and is acquired by the processing unit 106. In a case where the information processing apparatus 100 includes the learning model creation apparatus 200, the processing unit 106 acquires the trained model 107 from the learning model creation apparatus 200.

All or a part of the input unit 202, the reception unit 204, the processing unit 206, and the output unit 208 are functional units realized by a processor such as a CPU executing a program stored in the storage unit 210 (hereinafter, referred to as software functional units).

All or a part of the input unit 202, the reception unit 204, the processing unit 206, and the output unit 208 may be realized by hardware such as an LSI, an ASIC, or an FPGA, or may be realized by a combination of the software functional units and the hardware.

### (Operation of Learning Model Creation Apparatus 200)

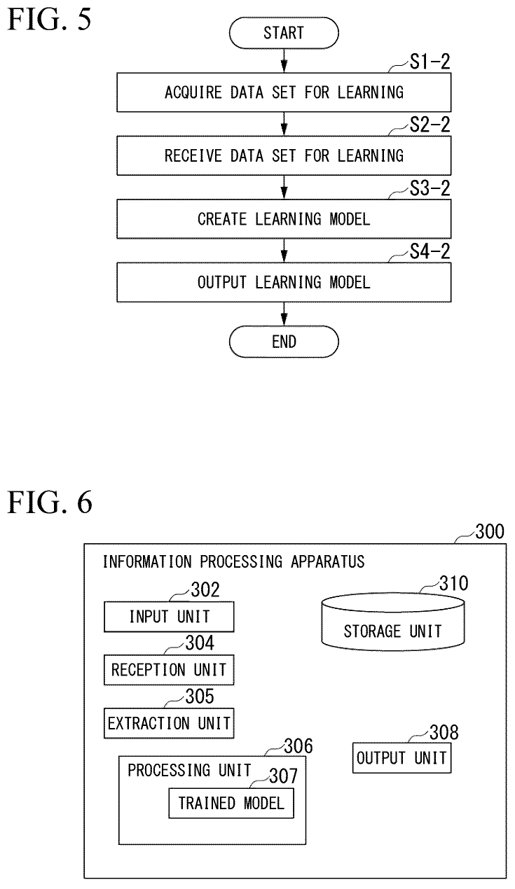

FIG. 5 is a flowchart showing an example of an operation of the learning model creation apparatus according to the present embodiment.

### (Step S1-2)

The input unit 202 acquires the data set for learning.

### (Step S2-2)

The reception unit 204 acquires the data set for learning from the input unit 202, and receives the acquired data set for learning.

### (Step S3-2)

The processing unit 206 acquires the data set for learning from the reception unit 204. The processing unit 206 calculates, for all pairs of the input sample and the output sample included in the data set for learning, the error between the output value output from the output layer by inputting the input sample to the input layer of the learning model 207 and the output sample (supervised data) corresponding to the input sample, and changes the parameter of the learning model 207 (trains the learning model 207) such that the error is as small as possible.

### (Step S4-2)

The output unit 208 acquires the learning model 207 from the processing unit 206. The output unit 208 outputs the acquired learning model 207.

In the above embodiment, the trained model 107 may be obtained by performing machine learning on a relationship between information indicating at least one of an intraocular pressure, an inter-pupillary distance, a height, or a corneal curvature radius and the information indicating the sphericity of the eye, in addition to the fundus image information of the eye.

In this case, in the information processing apparatus 100, the processing unit 106 may input, to the trained model 107, the fundus image information of the eye and the information indicating at least one of the intraocular pressure, the inter-pupillary distance, the height, or the corneal curvature radius to acquire the information indicating the sphericity of the eye, which is output by the trained model 107, for the input fundus image information of the eye and information indicating at least one of the intraocular pressure, the inter-pupillary distance, the height, or the corneal curvature radius.

In the above embodiment, the learning model creation apparatus 200 may create the trained model 107 by training the learning model (model that is the source of the trained model 107), using a data set for learning in which the information indicating at least one of the intraocular pressure, the inter-pupillary distance, the height, or the corneal curvature radius and the information indicating the sphericity of the eye are used as the input samples and the information indicating the sphericity of the eye of the subject is used as the output sample, in addition to the fundus image information of the eye of the subject as the model target.

In the above embodiment, the trained model 107 may be configured to include a first trained model obtained by performing machine learning on a relationship between the fundus image information of a right eye and the information indicating the sphericity of the right eye, and a second trained model obtained by performing machine learning on a relationship between the fundus image information of a left eye and the information indicating the sphericity of the left eye.

In this case, in the information processing apparatus 100, the processing unit 106 may input, to the first trained model, fundus coordinate information of the right eye included in the fundus image information of the eye to acquire the information indicating the sphericity of the right eye output by the first trained model for the input fundus image information of the right eye. Further, the processing unit 106 may input, to the second trained model, the fundus coordinate information of the left eye included in the fundus image information of the eye to acquire the information indicating the sphericity of the left eye output by the second trained model for the input fundus image information of the left eye.

In the above embodiment, the learning model creation apparatus 200 may create the trained model 107 including the first trained model and the second trained model by training each of the first learning model (model that is a source of the first trained model) and the second learning model (model that is a source of the second trained model), using a data set for learning in which the fundus image information of the right eye of the subject as the model target is used as the input sample and the information indicating the sphericity of the right eye of the subject is used as the output sample, and a data set for learning in which the fundus image information of the left eye of the subject as the model target is used as the input sample and the information indicating the sphericity of the left eye of the subject is used as the output sample.

In the above embodiment, the trained model 107 may be obtained by performing machine learning on a relationship between the fundus image information of the eye and information indicating an astigmatism power of the eye and information indicating an astigmatism axis thereof.

In this case, in the information processing apparatus 100, the processing unit 106 may input, to the trained model 107, the fundus image information of the eye to acquire the information indicating the astigmatism power of the eye and the information indicating the astigmatism axis thereof, which are output by the trained model 107, for the input fundus image information of the eye.

In the above embodiment, the learning model creation apparatus 200 may create the trained model 107 by training the learning model (model that is the source of the trained model 107), using a data set for learning in which the fundus image information of the eye of the subject as the model target is used as the input sample and the information indicating the astigmatism power of the eye of the subject and the information indicating the astigmatism axis thereof are used as the output samples.

In the above embodiment, the trained model 107 may be configured to include the first trained model obtained by performing machine learning on a relationship between the fundus image information of the right eye and the information indicating the astigmatism power of the right eye and the information indicating the astigmatism axis thereof, and the second trained model obtained by performing machine learning on a relationship between the fundus image information of the left eye and the information indicating the astigmatism power of the left eye and the information indicating the astigmatism axis thereof.

In this case, in the information processing apparatus 100, the processing unit 106 may input, to the first trained model, the fundus coordinate information of the right eye included in the fundus image information of the eye to acquire the information indicating the astigmatism power of the right eye and the information indicating the astigmatism axis thereof, which are output by the first trained model, for the input fundus image information of the right eye. Further, the processing unit 106 may input, to the second trained model, the fundus coordinate information of the left eye included in the fundus image information of the eye to acquire the information indicating the astigmatism power of the left eye and the information indicating the astigmatism axis thereof, which are output by the second trained model, for the input fundus image information of the left eye.

In the above embodiment, the learning model creation apparatus 200 may create the trained model 107 including the first trained model and the second trained model by training each of the first learning model (model that is the source of the first trained model) and the second learning model (model that is the source of the second trained model), using a data set for learning in which the fundus image information of the right eye of the subject as the model target is used as the input sample and the information indicating the astigmatism power of the right eye of the subject and the information indicating the astigmatism axis thereof are used as the output samples, and a data set for learning in which the fundus image information of the left eye of the subject as the model target is used as the input sample and the information indicating the astigmatism power of the left eye of the subject and the information indicating the astigmatism axis thereof are used as the output samples.

In the above embodiment, the trained model 107 may be obtained by performing machine learning on a relationship between the information indicating the astigmatism power of the eye and the information indicating the astigmatism axis thereof, in addition to the fundus image information of the eye and the information indicating the sphericity of the eye.

In this case, in the information processing apparatus 100, the processing unit 106 may input, to the trained model 107, the fundus image information of the eye to acquire the information indicating the sphericity of the eye, the information indicating the astigmatism power of the eye, and the information indicating the astigmatism axis thereof, which are output by the trained model 107, for the input fundus image information of the eye.

In the above embodiment, the learning model creation apparatus 200 may create the trained model 107 by training the learning model (model that is the source of the trained model 107), using a data set for learning in which the fundus image information of the eye of the subject as the model target is used as the input sample, and the information indicating the sphericity of the eye of the subject, the information indicating the astigmatism power, and the information indicating the astigmatism axis are used as the output samples.

In the above embodiment, the trained model 107 may be configured to include the first trained model obtained by performing machine learning on a relationship between the fundus image information of the right eye and the information indicating the sphericity of the right eye, the information indicating the astigmatism power, and the information indicating the astigmatism axis, and the second trained model obtained by performing machine learning on a relationship between the fundus image information of the left eye and the information indicating the sphericity of the left eye, the information indicating the astigmatism power, and information indicating the astigmatism axis.

In this case, in the information processing apparatus 100, the processing unit 106 may input, to the first trained model, the fundus coordinate information of the right eye included in the fundus image information of the eye to acquire the information indicating the sphericity of the right eye, the information indicating the astigmatism power, and the information indicating the astigmatism axis, which are output by the first trained model, for the input fundus image information of the right eye. Further, the processing unit 106 may input, to the second trained model, the fundus coordinate information of the left eye included in the fundus image information of the eye to acquire the information indicating the sphericity of the left eye, the information indicating the astigmatism power, and the information indicating the astigmatism axis, which are output by the second trained model, for the input fundus image information of the left eye.

In the above embodiment, the learning model creation apparatus 200 may create the trained model 107 including the first trained model and the second trained model by training each of the first learning model (model that is the source of the first trained model) and the second learning model (model that is the source of the second trained model), using a data set for learning in which the fundus image information of the right eye of the subject as the model target is used as the input sample and the information indicating the sphericity of the right eye of the subject, the information indicating the astigmatism power, and the information indicating the astigmatism axis are used as the output samples, and a data set for learning in which the fundus image information of the left eye of the subject as the model target is used as the input sample and the information indicating the sphericity of the left eye of the subject, the information indicating the astigmatism power, and the information indicating the astigmatism axis are used as the output samples.

With the information processing apparatus according to the present embodiment, the information processing apparatus 100 can receive the fundus image information of the subject, and acquire the information indicating the sphericity of the eye of the subject based on the trained model 107, which is obtained by performing machine learning on the relationship between the fundus image information of the eye and the information indicating the sphericity of the eye, and the received fundus image information of the subject. Therefore, it is possible to acquire the information indicating the sphericity of the eye, for the subject. With the use of the acquired information indicating the sphericity of the eye, it is possible to predict a myopia of the subject. A fundus image is acquired for an examination of diabetes or glaucoma in a health checkup or the like, and it is possible to know the sphericity in the examination, which is beneficial.

With the information processing apparatus according to the present embodiment, the information processing apparatus 100 can receive the fundus image information of the subject, and acquire the information indicating the sphericity of the right eye of the subject and the information indicating the sphericity of the left eye thereof based on the first trained model obtained by performing machine learning on the relationship between the fundus image information of the right eye and the information indicating the sphericity of the right eye, the second trained model obtained by performing machine learning on the relationship between the fundus image information of the left eye and the information indicating the sphericity of the left eye, and the received fundus image information of the subject. Therefore, it is possible to acquire the information indicating the sphericity of the right eye and the information indicating the sphericity of the left eye for the subject.

With the learning model creation apparatus according to the present embodiment, the learning model creation apparatus 200 can create the learning model 207 by performing machine learning on the relationship between the fundus image information of the eye and the information indicating the sphericity of the eye, using the fundus image information of the eye as an explanatory variable and the information indicating the sphericity of the eye as a response variable, based on the data set for learning.

With the learning model creation apparatus according to the present embodiment, the learning model creation apparatus 200 can create the first learning model by performing machine learning on the relationship between the fundus image information of the right eye and the information indicating the sphericity of the right eye, using the fundus image information of the right eye as the explanatory variable and the information indicating the sphericity of the right eye as the response variable, based on the data set for learning. Further, the learning model creation apparatus 200 can create the second learning model by performing machine learning on the relationship between the fundus image information of the left eye and the information indicating the sphericity of the left eye, using the fundus image information of the left eye as the explanatory variable and the information indicating the sphericity of the left eye as the response variable, based on the data set for learning.

### Modification Example 1 of Embodiment

An information processing apparatus 300 according to Modification Example 1 of the embodiment will be described.

FIG. 6 is a diagram showing an example of an information processing apparatus according to Modification Example 1 of the embodiment.

The information processing apparatus 300 according to Modification Example 1 of the embodiment is different from the information processing apparatus 100 of the embodiment in that the fundus image of a predetermined region including the macula and the optic nerve head is extracted, from the fundus image, based on the fundus image information of the subject.

The information processing apparatus 300 is formed by an apparatus such as a personal computer, a server, a smartphone, a tablet computer, or a computer for industry. The information processing apparatus 300 includes an input unit 302, a reception unit 304, an extraction unit 305, a processing unit 306, an output unit 308, and a storage unit 310.

Since the input unit 102, the reception unit 104, and the output unit 108 can each be applied as the input unit 302, the reception unit 304, and the output unit 308, a description thereof will be omitted.

The extraction unit 305 acquires, from the reception unit 104, the subject identification information and the fundus image information of the eye, and extracts, from the fundus image, the fundus image of the predetermined region including the macula and the optic nerve head based on the acquired fundus image information of the eye. The fundus image of the predetermined region is a region narrower than the fundus image acquired from the reception unit 104.

The processing unit 306 acquires, from the extraction unit 305, the subject identification information and the fundus image information of the predetermined region. The processing unit 306 includes a trained model 307.

The trained model 307 is obtained by performing machine learning on the relationship between the fundus image information of the eye and the information indicating the sphericity of the eye.

The processing unit 306 inputs, to the trained model 307, the acquired fundus image information of the predetermined region to acquire the information indicating the sphericity of the eye.

All or a part of the input unit 302, the reception unit 304, the extraction unit 305, the processing unit 306, and the output unit 308 are functional units realized by a processor such as a CPU executing a program stored in the storage unit 310 (hereinafter, referred to as software functional units). All or a part of the input unit 302, the reception unit 304, the extraction unit 305, the processing unit 306, and the output unit 308 may be realized by hardware such as an LSI, an ASIC, or an FPGA, or may be realized by a combination of the software functional units and the hardware.

### (Operation of Information Processing Apparatus 300)

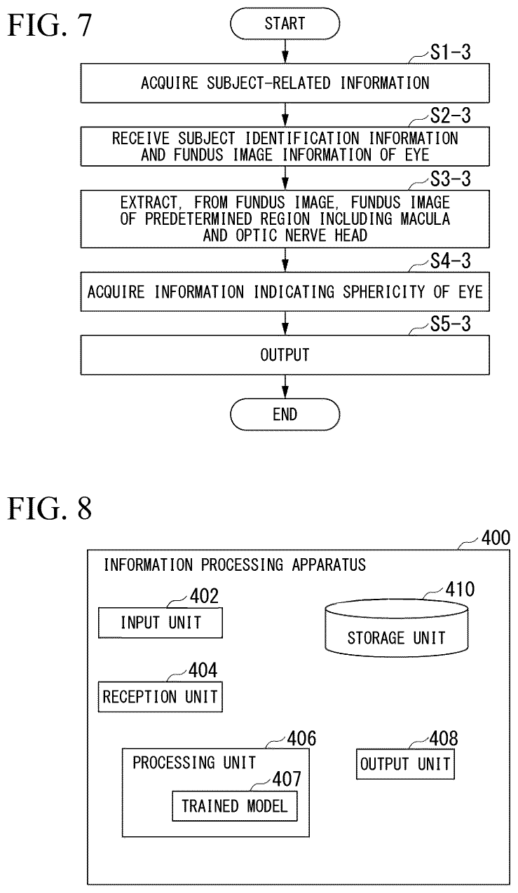

FIG. 7 is a flowchart showing an example of an operation of the information processing apparatus according to Modification Example 1 of the embodiment.

### (Step S1-3)

The input unit 302 acquires the subject-related information.

### (Step S2-3)

The reception unit 304 acquires the subject-related information from the input unit 302. The reception unit 304 receives the subject identification information, which is included in the acquired subject-related information, and the fundus image information of the eye.

### (Step S3-3)

The extraction unit 305 acquires, from the reception unit 304, the subject identification information and the fundus image information of the eye, and extracts, from the fundus image of the eye, the fundus image of the eye of the predetermined region including the macula and the optic nerve head based on the acquired fundus image information of the eye.

### (Step S4-3)

The processing unit 306 acquires, from the extraction unit 305, the subject identification information and the fundus image information of the eye of the predetermined region. The processing unit 306 inputs, to the trained model 307, the acquired fundus image information of the eye of the predetermined region to acquire the information indicating the sphericity of the eye, which is output by the trained model 307, for the input fundus image information of the eye of the predetermined region.

### (Step S5-3)

The output unit 308 acquires, from the processing unit 306, the subject identification information and the information indicating the sphericity of the eye. The output unit 308 outputs the acquired subject identification information and information indicating the sphericity of the eye.

In Modification Example 1 of the above embodiment, the processing unit 306 inputs the fundus image information of the eye of the subject to the trained model 307 in which the fundus image information of the eye of the subject whose sphericity of the eye is actually acquired is already stored to acquire the information indicating the sphericity of the eye of the subject. Hereinafter, the fundus image information of the eye related to the generation of the trained model 107 (subject whose sphericity of the eye is actually acquired and whose fundus image information of the eye is already stored) will be referred to as the model target.

Since the method of generating the trained model 107 described above can be applied as a method of generating the trained model 307, a description thereof will be omitted.

In Modification Example 1 of the above embodiment, the trained model 307 may be configured to include the first trained model obtained by performing machine learning on the relationship between the fundus image information of the right eye and the information indicating the sphericity of the right eye, and the second trained model obtained by performing machine learning on the relationship between the fundus image information of the left eye and the information indicating the sphericity of the left eye.

In this case, the processing unit 306 may input, to the first trained model, the fundus coordinate information of the right eye, which is included in the fundus image of the predetermined region including the macula and the optic nerve head extracted from the fundus image, to acquire the information indicating the sphericity of the right eye, which is output by the first trained model, for the input fundus image information of the right eye. Further, the processing unit 306 may input, to the second trained model, the fundus coordinate information of the left eye, which is included in the fundus image of the predetermined region including the macula and the optic nerve head extracted from the fundus image, to acquire the information indicating the sphericity of the left eye, which is output by the second trained model, for the input fundus image information of the left eye.

With the information processing apparatus according to Modification Example 1 of the embodiment, the information processing apparatus 300 can extract, from the fundus image, the fundus image of the predetermined region including the macula and the optic nerve head based on the fundus image information of the subject, and acquire the information indicating the sphericity of the eye of the subject based on the trained model and the extracted fundus image information of the predetermined region. The information indicating the sphericity of the eye of the subject can be acquired based on the fundus image of the predetermined region including the macula and the optic nerve head extracted from the fundus image. Therefore, it is possible to improve the accuracy as compared with a case where the information indicating the sphericity of the eye of the subject is acquired based on the fundus image.

### Modification Example 2 of Embodiment

An information processing apparatus 400 according to Modification Example 2 of the embodiment will be described.

FIG. 8 is a diagram showing an example of an information processing apparatus according to Modification Example 2 of the embodiment.

The information processing apparatus 400 according to Modification Example 2 of the embodiment is different from the information processing apparatus 100 of the embodiment in that the information indicating the ocular axial length of the subject is acquired, based on a trained model obtained by further performing machine learning on the relationship between the fundus image of the eye and the ocular axial length of the eye, in addition to the sphericity of the eye, and the received fundus image information of the subject.

The information processing apparatus 400 is formed by an apparatus such as a personal computer, a server, a smartphone, a tablet computer, or a computer for industry. The information processing apparatus 400 includes an input unit 402, a reception unit 404, a processing unit 406, an output unit 408, and a storage unit 410.

Since the input unit 102 and the reception unit 104 can each be applied as the input unit 402 and the reception unit 304, a description thereof will be omitted.

The processing unit 406 acquires, from the reception unit 404, the subject identification information and the fundus image information of the eye. The processing unit 406 includes a trained model 407.

The trained model 407 is obtained by performing machine learning on the relationship between the fundus image information of the eye, and the information indicating the sphericity of the eye and the information indicating the ocular axial length of the eye.

The processing unit 406 inputs, to the trained model 407, the fundus image information to acquire the information indicating the sphericity of the eye and the information indicating the ocular axial length of the eye.

All or a part of the input unit 402, the reception unit 404, the processing unit 406, and the output unit 408 are functional units realized by a processor such as a CPU executing a program stored in the storage unit 410 (hereinafter, referred to as software functional units). All or a part of the input unit 402, the reception unit 404, the processing unit 406, and the output unit 408 may be realized by hardware such as an LSI, an ASIC, or an FPGA, or may be realized by a combination of the software functional units and the hardware.

### (Operation of Information Processing Apparatus 400)

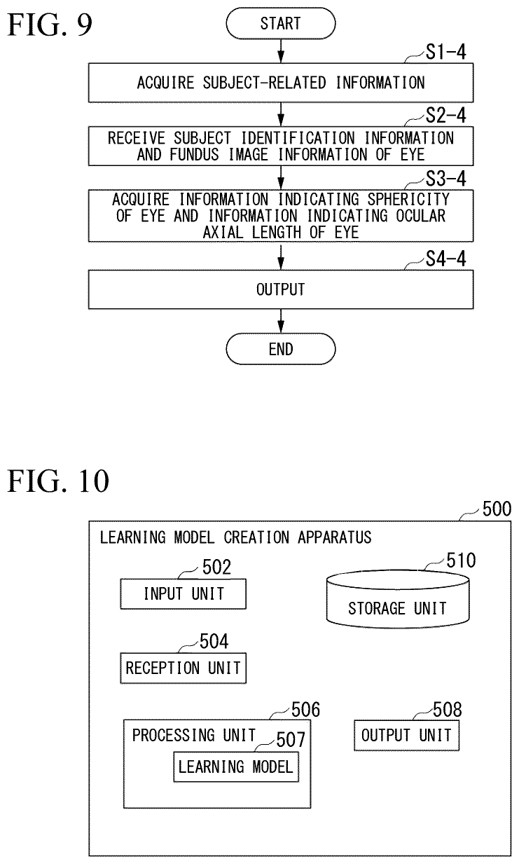

FIG. 9 is a flowchart showing an example of an operation of the information processing apparatus according to Modification Example 2 of the embodiment.

### (Step S1-4)

The input unit 402 acquires the subject-related information.

### (Step S2-4)

The reception unit 404 acquires the subject-related information from the input unit 402. The reception unit 404 receives the subject identification information, which is included in the acquired subject-related information, and the fundus image information of the eye.

### (Step S3-4)

The processing unit 406 acquires, from the reception unit 404, the subject identification information and the fundus image information of the eye. The processing unit 406 inputs, to the trained model 407, the acquired fundus image information of the eye to acquire the information indicating the sphericity of the eye and the information indicating the ocular axial length of the eye, which are output by the trained model 407, for the input fundus image information of the eye.

### (Step S4-4)

The output unit 408 acquires, from the processing unit 406, the subject identification information, the information indicating the sphericity of the eye, and the information indicating the ocular axial length of the eye. The output unit 408 outputs the acquired subject identification information, information indicating the sphericity of the eye, and information indicating the ocular axial length of the eye.

In the modification example of the above embodiment, the processing unit 406 inputs the fundus image information of the eye of the subject to the trained model 407 in which the fundus image information of the eye of the subject whose sphericity of the eye and the ocular axial length of the eye are actually acquired is already stored to acquire the information indicating the sphericity of the eye of the subject and the information indicating the ocular axial length of the eye thereof. Hereinafter, the fundus image information of the eye related to the generation of the trained model 407 (subject whose sphericity of the eye and the ocular axial length are actually acquired and whose fundus image information of the eye is already stored) will be referred to as the model target.

The generation of the trained model 407 will be described. The trained model 407 is created by the learning model creation apparatus. That is, the learning model creation apparatus creates the trained model 407. The information processing apparatus 400 may include the learning model creation apparatus. That is, the information processing apparatus 400 may create the trained model 407.

FIG. 10 is a diagram showing an example of a learning model creation apparatus according to Modification Example 2 of the embodiment. A learning model creation apparatus 500 according to the modification example of the embodiment is formed by an apparatus such as a personal computer, a server, a smartphone, a tablet computer, or a computer for industry.

The learning model creation apparatus 500 creates the trained model 407 by training a learning model (model that is a source of the trained model 407), using a data set for learning in which the fundus image information of the eye of the subject as the model target is used as the input sample and the information indicating the sphericity of the eye of the subject and the information indicating the ocular axial length of the eye are used as the output samples.

For example, the learning model creation apparatus 500 uses an algorithm such as the CNN, the RNN, the LSTM, the random forest, the SVM, or the neural network to construct the trained model 407. The input sample is data input to an input layer during the training of the learning model. The output sample is data (supervised data) that is a correct answer to be compared with an output value output from an output layer during the training of the learning model.

The learning model creation apparatus 500 includes an input unit 502, a reception unit 504, a processing unit 506, an output unit 508, and a storage unit 510.

The input unit 202 can be applied as the input unit 502. The data set for learning is input to the input unit 502. The reception unit 504 acquires the data set for learning from the input unit 502. The input sample and the output sample are paired, and the data set for learning is configured of a plurality of pairs.

The processing unit 506 calculates, for all pairs, an error between the output value output from the output layer by inputting the input sample to the input layer of the learning model 507 and the output sample (supervised data) corresponding to the input sample, and changes a parameter of the learning model 507 (trains the learning model 507) such that the error is as small as possible to create the trained model 407. For example, the processing unit 506 may perform the transfer learning, using the learning model 507 on which pre-training using data such as ImageNet is performed.

The trained model 407 created as described above is received, from the output unit 508, by the information processing apparatus 400 via a network or a medium, and is acquired by the processing unit 406. In a case where the information processing apparatus 400 includes the learning model creation apparatus 500, the processing unit 406 acquires the trained model 407 from the learning model creation apparatus 500.

All or a part of the processing unit 506 is, for example, a functional unit realized by a processor such as a CPU executing a program stored in the storage unit 510 (hereinafter, referred to as software functional unit). All or a part of the processing unit 506 may be realized by hardware such as an LSI, an ASIC, or an FPGA, or may be realized by a combination of the software functional unit and the hardware.

### (Operation of Learning Model Creation Apparatus 500)

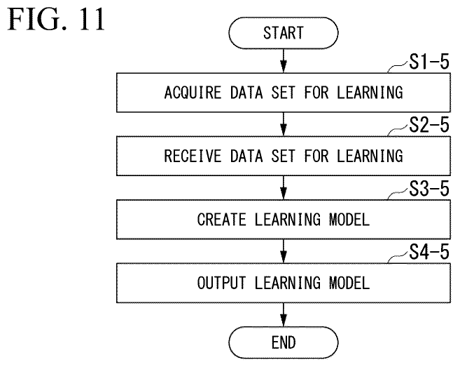

FIG. 11 is a flowchart showing an example of an operation of the learning model creation apparatus according to Modification Example 2 of the embodiment.

### (Step S1-5)

The input unit 502 acquires the data set for learning.

### (Step S2-5)

The reception unit 504 acquires the data set for learning from the input unit 502. The reception unit 504 receives the acquired data set for learning.

### (Step S3-5)

The processing unit 506 acquires the data set for learning from the reception unit 504. The processing unit 506 calculates, for all pairs of the input sample and the output sample included in the data set for learning, the error between the output value output from the output layer by inputting the input sample to the input layer of the learning model 507 and the output sample (supervised data) corresponding to the input sample, and changes the parameter of the learning model 507 (trains the learning model 507) such that the error is as small as possible.

### (Step S4-5)

The output unit 508 acquires the learning model 507 from the processing unit 506. The output unit 508 outputs the acquired learning model 507.

With the information processing apparatus according to Modification Example 2 of the embodiment, the information processing apparatus 400 can acquire the information indicating the sphericity of the eye of the subject and the information indicating the ocular axial length of the eye, based on a trained model obtained by further performing machine learning on the relationship between the fundus image of the eye and the ocular axial length of the eye, in the information processing apparatus 100 and the received fundus image information of the subject. Therefore, it is possible to acquire the information indicating the ocular axial length of the eye, in addition to the information indicating the sphericity of the eye, for the subject. With the use of the acquired information indicating the sphericity of the eye and ocular axial length of the eye, it is possible to predict the myopia of the subject. The fundus image is acquired for the examination of diabetes or glaucoma in a health checkup or the like, and it is possible to know the sphericity and the ocular axial length of the eye in the examination, which is beneficial.

With the learning model creation apparatus according to the present embodiment, the learning model creation apparatus 500 can create the learning model by receiving, in the learning model creation apparatus 200, the data set for learning in which the fundus image information of the eye is included as learning data and the information indicating the ocular axial length of the eye is further included as the supervised data, and performing machine learning on the relationship between the fundus image information of the eye, and the information indicating the sphericity of the eye and the information indicating the ocular axial length of the eye, using the fundus image information of the eye as the explanatory variable and the information indicating the sphericity of the eye and the information indicating the ocular axial length of the eye as the response variable, based on the received data set for learning.

### Configuration Example

As one configuration example, an information processing apparatus includes a reception unit configured to receive fundus image information of a subject, a processing unit configured to acquire information indicating a sphericity of an eye of the subject based on a trained model, which is obtained by performing machine learning on a relationship between fundus image information of an eye and information indicating a sphericity of the eye, and the fundus image information of the subject received by the reception unit, and an output unit configured to output the information indicating the sphericity of the eye of the subject acquired by the processing unit.

As one configuration example, the processing unit acquires information indicating an ocular axial length of the eye of the subject based on a trained model, which is obtained by further performing machine learning on a relationship between a fundus image of the eye and an ocular axial length of the eye, and the fundus image information of the subject received by the reception unit, and the output unit outputs the information indicating the ocular axial length of the eye of the subject acquired by the processing unit.

As one configuration example, the information processing apparatus further includes an extraction unit configured to extract, from a fundus image, a fundus image of a predetermined region including a macula and an optic nerve head based on the fundus image information of the subject. The processing unit acquires the information indicating the sphericity of the eye of the subject, based on the trained model and fundus image information of the predetermined region extracted by the extraction unit.

As one configuration example, the trained model includes a first trained model obtained by performing machine learning on a relationship between fundus image information of a right eye and information indicating a sphericity of the right eye, and a second trained model obtained by performing machine learning on a relationship between fundus image information of a left eye and information indicating a sphericity of the left eye.

Although the embodiments of the present invention and the modification examples of the embodiments have been described in detail with reference to the drawings, the specific configuration is not limited to the embodiments and the modification examples of the embodiments, and design changes and the like within a range not departing from the scope of the present invention are also included. For example, Modification Example 1 of the embodiment and Modification Example 2 of the embodiment may be combined.

Further, a computer program for realizing the functions of the information processing apparatus 100, the information processing apparatus 300, the information processing apparatus 400, the learning model creation apparatus 200, and the learning model creation apparatus 500 described above may be recorded on a computer-readable recording medium, and the program recorded on the recording medium may be loaded into a computer system and executed. The term “computer system” described herein may include an OS and hardware such as peripheral devices.

Further, the term “computer-readable recording medium” refers to a storage device such as a writable non-volatile memory such as a flexible disk, a magneto-optical disk, a ROM, or a flash memory, a portable medium such as a digital versatile disk (DVD), or a hard disk built in the computer system.

Furthermore, the term “computer-readable recording medium” also includes a medium that maintains the program for a certain time, such as a volatile memory (for example, dynamic random access memory (DRAM)) in the computer system as a server or a client in a case where the program is transmitted via a network such as the Internet or a communication line such as a telephone line.

Further, the program may be transmitted, to another computer system, from a computer system in which this program is stored in a storage device or the like, via a transmission medium or by a transmission wave in the transmission medium. The term “transmission medium” that transmits the program refers to a medium having a function of transmitting information, such as a network (communication network) such as the Internet or a communication line such as a telephone line.

Further, the above program may realize some of the above functions. Furthermore, the above program may be a so-called difference file (difference program) that can realize the above functions in combination with the program already recorded in the computer system.

While preferred embodiments of the invention have been described and illustrated above, it should be understood that these are exemplary of the invention and are not to be considered as limiting. Additions, omissions, substitutions, and other modifications can be made without departing from the scope of the invention. Accordingly, the invention is not to be considered as being limited by the foregoing description and is only limited by the scope of the appended claims.

### EXPLANATION OF REFERENCES

100, 300, 400: information processing apparatus
102, 302, 402: input unit
104, 304, 404: reception unit
305: extraction unit
106, 306, 406: processing unit
107, 307, 407: trained model
108, 308, 408: output unit
110, 310, 410: storage unit
200, 500: learning model creation apparatus
202, 502: input unit
204, 504: reception unit
206, 506: processing unit
207, 507: learning model
208, 508: output unit
210, 510: storage unit

What is claimed is:

1. An information processing apparatus comprising:
a reception unit configured to receive fundus image information of a subject;
a processing unit configured to acquire information indicating a sphericity of an eye of the subject based on a trained model, which is obtained by performing machine learning on a relationship between fundus image information of an eye and information indicating a sphericity of the eye, and the fundus image information of the subject received by the reception unit; and
an output unit configured to output the information indicating the sphericity of the eye of the subject acquired by the processing unit.

2. The information processing apparatus according to claim 1,
wherein the processing unit acquires information indicating an ocular axial length of the eye of the subject based on a trained model, which is obtained by further performing machine learning on a relationship between a fundus image of the eye and an ocular axial length of the eye, and the fundus image information of the subject received by the reception unit, and
the output unit outputs the information indicating the ocular axial length of the eye of the subject acquired by the processing unit.

3. The information processing apparatus according to claim 1, further comprising:
an extraction unit configured to extract, from a fundus image, a fundus image of a predetermined region including a macula and an optic nerve head based on the fundus image information of the subject,
wherein the processing unit acquires the information indicating the sphericity of the eye of the subject, based on the trained model and fundus image information of the predetermined region extracted by the extraction unit.

4. The information processing apparatus according to claim 1,
wherein the trained model includes a first trained model obtained by performing machine learning on a relationship between fundus image information of a right eye and information indicating a sphericity of the right eye, and a second trained model obtained by performing machine learning on a relationship between fundus image information of a left eye and information indicating a sphericity of the left eye.

5. A learning model creation apparatus comprising:
a reception unit configured to receive a data set for learning in which fundus image information of an eye is included as learning data and information indicating a sphericity of the eye is included as supervised data;
a processing unit configured to create a learning model by performing machine learning on a relationship between the fundus image information of the eye and the information indicating the sphericity of the eye, using the fundus image information of the eye as an explanatory variable and the information indicating the sphericity of the eye as a response variable, based on the data set for learning received by the reception unit; and
an output unit configured to output the learning model created by the processing unit.

6. The learning model creation apparatus according to claim 5,
wherein the reception unit receives a data set for learning in which the fundus image information of the eye is included as the learning data and information indicating an ocular axial length of the eye is further included as the supervised data, and
the processing unit creates a learning model by performing machine learning on a relationship between the fundus image information of the eye and the information indicating the ocular axial length of the eye, using the fundus image information of the eye as the explanatory variable and the information indicating the ocular axial length of the eye as the response variable, based on the data set for learning received by the reception unit.

7. An information processing method executed by a computer, the method comprising:
receiving fundus image information of a subject;
acquiring information indicating a sphericity of an eye of the subject based on a trained model, which is obtained by performing machine learning on a relationship between fundus image information of an eye and information indicating a sphericity of the eye, and the received fundus image information of the subject; and
outputting the acquired information indicating the sphericity of the eye of the subject.

8. A learning model creation method executed by a computer, the method comprising:
receiving a data set for learning in which fundus image information of an eye is included as learning data and information indicating a sphericity of the eye is included as supervised data;
creating a learning model by performing machine learning on a relationship between the fundus image information of the eye and the information indicating the sphericity of the eye, using the fundus image information of the eye as an explanatory variable and the information indicating the sphericity of the eye as a response variable, based on the received data set for learning; and
outputting the created learning model.

9. A program causing a computer to:
receive fundus image information of a subject;
acquire information indicating a sphericity of an eye of the subject based on a trained model, which is obtained by performing machine learning on a relationship between fundus image information of an eye and information indicating a sphericity of the eye, and the received fundus image information of the subject; and
output the acquired information indicating the sphericity of the eye of the subject.

10. A program causing a computer to:
receive a data set for learning in which fundus image information of an eye is included as learning data and information indicating a sphericity of the eye is included as supervised data;
create a learning model by performing machine learning on a relationship between the fundus image information of the eye and the information indicating the sphericity of the eye, using the fundus image information of the eye as an explanatory variable and the information indicating the sphericity of the eye as a response variable, based on the received data set for learning; and
output the created learning model.
